(19) United States
(12) Patent Application Publication
(10) Pub. No.: US 2026/0306259 A1
(43) Pub. Date: Oct. 8, 2026

(54) SPINDLE WITH LEAFLET PROTRUSION DAMPENERS

(71) Applicant: Medtronic, Inc., Minneapolis, MN (US)

(72) Inventors: Christopher James MCCABE, Oranmore (IE); Timothy Desmond FARRELL, Tralee, KY (US)

(21) Appl. No.: 19/479,047

(22) PCT Filed: Apr. 23, 2024
(86) PCT No.: PCT/IB2024/053954

§ 371 Date: Oct. 27, 2025

Related U.S. Application Data
(60) Provisional application No. 63/498,827, filed on Apr. 28, 2023.

(51) Int. Cl. A61F 2/24

(52) U.S. Cl. CPC A61F 2/2436; A61F 2/2418; A61F 2250/0014

(57) ABSTRACT

A delivery assembly for percutaneously delivering a transcatheter heart valve prosthesis into a vasculature of a patient includes a capsule and a spindle. The capsule is configured to constrain the transcatheter heart valve prosthesis therein. The spindle includes a spindle body coupled to a shaft disposed within the capsule, and a dampening arm having a first end and a second end. The first being of the dampening arm is coupled to the spindle body and the second end of the dampening arm is configured to extend between an interior surface of a frame and a leaflet of the transcatheter heart valve prosthesis to minimize leaflet protrusion through cells of a frame of the transcatheter heart valve prosthesis.

### CROSS REFERENCE TO RELATED APPLICATION

This application claims the benefit of U.S. Provisional Patent Application Ser. No. 63/498,827, filed Apr. 28, 2023, the entire content of which is incorporated herein by reference.

### FIELD OF THE INVENTION

The present invention relates to a spindle extension that minimizes leaflet protrusion and leaflet damage during the loading/recapture of a transcatheter heart valve prosthesis into a delivery catheter and deployment of the transcatheter heart valve prosthesis.

### BACKGROUND

Diseases associated with heart valves, such as those caused by damage or a defect, can include stenosis and valvular insufficiency or regurgitation. For example, valvular stenosis causes the valve to become narrowed and hardened which can prevent blood flow to a downstream heart chamber from occurring at the proper flow rate and may cause the heart to work harder to pump the blood through the diseased valve. Valvular insufficiency or regurgitation occurs when the valve does not close completely, allowing blood to flow backwards, thereby causing the heart to be less efficient. A diseased or damaged valve, which can be congenital, age-related, drug-induced, or in some instances, caused by infection, can result in an enlarged, thickened heart that loses elasticity and efficiency. Some symptoms of heart valve diseases can include weakness, shortness of breath, dizziness, fainting, palpitations, anemia and edema, and blood clots which can increase the likelihood of stroke or pulmonary embolism. Symptoms can often be severe enough to be debilitating and/or life threatening.

Heart valve prostheses have been developed for repair and replacement of diseased and/or damaged heart valves. Such heart valve prostheses can be percutaneously delivered and deployed at the site of the diseased heart valve through catheter-based delivery systems. Such heart valve prostheses generally include a frame or stent and a prosthetic valve mounted within the frame. Such heart valve prostheses are delivered in a radially compressed or crimped configuration so that the heart valve prosthesis can be advanced through the patient's vasculature. Once positioned at the treatment site, the heart valve prosthesis is expanded to engage tissue at the diseased heart valve region to, for instance, hold the heart valve prosthesis in position.

In some circumstances, when radially compressing/crimping a heart valve prosthesis into delivery catheter, recapturing a heart valve prosthesis into a delivery catheter after at least partial deployment thereof, and/or during deployment of heart valve prosthesis from a delivery catheter, leaflets of the prosthetic heart valve may get trapped/pinched between struts of the frame as the frame is radially compressed/crimped into the radially compressed configuration or as the frame is radially expanded to the radially expanded configuration. This may cause damage to the leaflets and affect performance and longevity of the heart valve prosthesis in vivo. The present disclosure relates to improvements in delivery catheters to minimize or prevent pinching of the leaflets of the heart valve prosthesis during loading, recapture, and/or deployment thereof.

### BRIEF SUMMARY OF THE INVENTION

In accordance with a first example hereof, a delivery assembly for percutaneously delivering a transcatheter heart valve prosthesis into a vasculature of a patient includes a capsule configured to constrain the transcatheter heart valve prosthesis therein, and a spindle. The spindle includes a spindle body coupled to a shaft disposed within the capsule, and a dampening arm having a first end and a second end. The first end of the dampening arm is coupled to the spindle body and the second end of the dampening arm is configured to extend between an interior surface of a frame and a leaflet of the transcatheter heart valve prosthesis to minimize leaflet protrusion through cells of the frame of the transcatheter heart valve prosthesis.

In a second example hereof, the delivery assembly according to any of the previous or subsequent examples hereof further comprises a transcatheter heart valve prosthesis, the transcatheter heart valve prosthesis including a frame and a valve structure coupled to the frame, wherein the valve structure includes a plurality of leaflets, and wherein with the transcatheter heart valve prosthesis radially compressed within the capsule, the dampening arm extends within the frame with the second end of the dampening arm disposed radially outside of a leaflet of the plurality of leaflets.

In a third example hereof, in the delivery assembly according to any of the previous or subsequent examples hereof, the frame of the transcatheter heart valve prosthesis includes access cells disposed adjacent the plurality of leaflets of the valve structure, wherein the access cells are larger than other cells of the frame.

In a fourth example hereof, in the delivery assembly according to any of the previous or subsequent examples hereof, the transcatheter heart valve prosthesis includes exactly three leaflets and exactly three access cells, and the spindle includes exactly three dampening arms.

In a fifth example hereof, in the delivery assembly according to any of the previous or subsequent examples hereof, the spindle body further includes a cavity configured to receive the first end of the dampening arm.

In a sixth example hereof, in the delivery assembly according to any of the previous or subsequent examples hereof, the spindle body includes exactly three cavities equidistantly spaced apart from one another around a circumference of the spindle body.

In a seventh example hereof, in the delivery assembly according to any of the previous or subsequent examples hereof, the cavity is disposed at a distal portion of the spindle body such that a proximal portion of the spindle body does not contain the cavity.

In an eighth example hereof, in the delivery assembly according to any of the previous or subsequent examples hereof, the spindle further comprises a locking collar configured to secure the first end of the dampening arm to the spindle body.

In a ninth example hereof, in the delivery assembly according to any of the previous or subsequent examples hereof, spindle includes a first configuration wherein the locking collar is longitudinally spaced from the cavity such that the cavity is open to receive the first end of the damping arm and the spindle includes a second configuration wherein the locking collar surrounds the cavity with the first end of the dampening arm disposed in the cavity to couple the dampening arm to the spindle body.

In a tenth example hereof, in the delivery assembly according to any of the previous or subsequent examples hereof, the dampening arm further includes a pad coupled to the second end thereof.

In an eleventh example hereof, in the delivery assembly according to any of the previous or subsequent examples hereof, the pad is substantially diamond-shaped.

In a twelfth example hereof, in the delivery assembly according to any of the previous or subsequent examples hereof, the pad is substantially the same size as the access cell of the transcatheter heart valve prosthesis.

In a thirteenth example hereof, in the delivery assembly according to any of the previous or subsequent examples hereof, the pad comprises a mesh material.

In a fourteenth example hereof, in the delivery assembly according to any of the previous or subsequent examples hereof, a first portion of the pad includes a covering material and a second portion of the pad is open to enable blood flow therethrough.

In a fifteenth example hereof, a method of delivering and deploying a transcatheter heart valve prosthesis to a treatment site within a vasculature of a patient comprises: advancing a delivery device to the treatment site with the transcatheter heart valve prosthesis radially compressed within a capsule of the delivery device, the transcatheter heart valve prosthesis including a frame and a valve structure coupled to the frame, wherein the valve structure includes a plurality of leaflets, wherein the delivery device includes a spindle coupled to a shaft of the delivery device disposed within the capsule, and a dampening arm extending from the spindle with a distal end of the dampening arm disposed between an interior surface of the frame and a leaflet of the plurality of leaflets of the transcatheter heart valve prosthesis; proximally retracting the capsule to partially release the transcatheter heart valve prosthesis from the capsule, wherein the spindle of the delivery device do not move relative to the transcatheter heart valve prosthesis such that the distal end of the dampening arm remains disposed between the interior surface of the frame and the leaflet; further proximally retracting the capsule further to fully deploy the transcatheter heart valve prosthesis; and proximally retracting the shaft and the spindle from the transcatheter heart valve prosthesis such that the dampening arm retracts from the transcatheter heart valve prosthesis.

In a sixteenth example hereof, the method according to any of the previous or subsequent examples hereof further comprises removing the delivery device from the vasculature of the patient.

In a seventeenth example hereof, the method according to any of the previous or subsequent examples hereof further comprises after proximally retracting the capsule to partially release the transcatheter heart valve prosthesis from the capsule and before further proximally retracting the capsule to fully release the transcatheter heart valve prosthesis, distally advancing the capsule to recapture the transcatheter heart valve prosthesis within the capsule, wherein during the distally advancing step, the distal end of the dampening arm is disposed between the leaflet and the interior surface of the frame to inhibit the leaflet from being pinched between struts of the frame during recapture of the transcatheter heart valve prosthesis.

In an eighteenth example hereof, in the method according to any of the previous or subsequent examples hereof, the transcatheter heart valve prosthesis includes exactly three leaflets, the spindle includes exactly three dampening arms, and wherein the second end of each dampening arm is disposed between a corresponding one of the leaflets and the frame.

In a nineteenth example hereof, in the method according to any of the previous or subsequent examples hereof, the dampening arm further comprises a pad coupled to the distal end of the dampening arm.

In a twentieth example hereof, in the method according to any of the previous or subsequent examples hereof, the pad includes a first portion and a second portion, wherein the first portion includes a covering material and the second portion is open to enable blood flow therethrough, wherein during the proximally retracting and further proximally retracting steps, the second portion of the pad enables blood flow therethrough.

In a twenty-first example hereof, a method of loading a transcatheter heart valve prosthesis into a capsule of a delivery device comprises: disposing a distal end of a dampening arm of a spindle between a leaflet of the transcatheter heart valve prosthesis and a frame of the transcatheter heart valve prosthesis; and radially compressing the transcatheter heart valve prosthesis within the capsule of the delivery device.

In a twenty-second example hereof, the method according to any of the previous or subsequent examples hereof further comprises coupling a proximal end of the dampening arm to a spindle body of the spindle.

In a twenty-third example hereof, in the method according to any of the previous or subsequent examples hereof, coupling the proximal end of the dampening arm to the spindle body is after disposing the distal end of the dampening arm between the leaflet and the frame.

In a twenty-fourth example hereof, in the method according to any of the previous or subsequent examples hereof, the spindle body comprises a cavity, and coupling the proximal end of the dampening arm of the spindle body comprises placing the proximal end of the dampening arm in the cavity and covering the cavity with the proximal end of the dampening arm disposed therein with a locking collar of the spindle.

In a twenty-fifth example hereof, in the method according to any of the previous or subsequent examples hereof, the locking collar is disposed over a first portion of the spindle body with the cavity open and covering the cavity comprises translating the locking collar to the cover the cavity.

In a twenty-sixth example hereof, in the method according to any of the previous or subsequent examples hereof, the spindle comprises exactly three dampening arms and the spindle body includes exactly three cavities equidistantly spaced apart from one another around a circumference of the spindle body.

In a twenty-seventh example hereof, the method according to any of the previous or subsequent examples hereof further comprises coupling the frame of the transcatheter heart valve prosthesis to the spindle.

In a twenty-eighth example hereof, in the method according to any of the previous or subsequent examples hereof, coupling the frame of the transcatheter heart valve prosthesis to the spindle comprises placing a paddle extending from an end of the frame into a paddle pocket of the spindle body.

In a twenty-ninth example hereof, in the method according to any of the previous or subsequent examples hereof, the dampening arm further includes a pad coupled to the distal end of the dampening arm.

In a thirtieth example hereof, in the method according to any of the previous or subsequent examples hereof, a portion the pad is disposed outside of an outer surface of the frame.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

The foregoing and other features and advantages of the present disclosure will be apparent from the following description of embodiments hereof as illustrated in the accompanying drawings. The accompanying drawings, which are incorporated herein and form a part of the specification, further serve to explain the principles of the present disclosure and to enable a person skilled in the pertinent art to make and use the embodiments of the present disclosure. The drawings may not be to scale.

FIG. 1A shows a side view of an example of a transcatheter heart valve prosthesis.

FIG. 1B shows an outflow view of the transcatheter heart valve prosthesis of FIG. 1A.

FIG. 2A is a schematic perspective view of a delivery assembly in a delivery configuration according to embodiments hereof.

FIG. 2B is a schematic perspective view of a delivery assembly of FIG. 2A in a deployed configuration according to embodiments hereof.

FIG. 3A is a schematic of a side cross-section of a distal portion of the delivery assembly of FIG. 2A in the delivery configuration, showing a spindle according to embodiments hereof.

FIG. 3B is a schematic of a side cross-section of a distal portion of the delivery assembly of FIG. 2A with a capsule of the delivery system partially retracted according to embodiments hereof.

FIG. 3C is a cross-section of the distal portion of the delivery assembly taken at line C-C of FIG. 3B, showing the spindle, a capsule and a transcatheter heart valve prosthesis according to embodiments hereof.

FIG. 3D is a schematic side view of a spindle before the dampening arms are secured to the spindle body using a locking collar according to embodiments hereof.

FIG. 3E is a cross-section of the spindle taken at line E-E of FIG. 3D according to embodiments hereof.

FIG. 3F is a schematic side view of the spindle of FIG. 3C after the dampening arms are secured to the spindle body using the locking collar according to embodiments hereof.

FIG. 3G is a cross-section of the spindle taken at line G-G of FIG. 3F according to embodiments hereof.

FIG. 3H is a flat, as-cut illustration of the transcatheter heart valve prosthesis partially disposed within the capsule of the delivery system and showing the dampening arms of the spindle according to embodiments hereof.

FIG. 3I is a schematic of a side cross-section of the distal portion of the delivery assembly of FIG. 3A with the transcatheter heart valve prosthesis deployed from the capsule of the delivery assembly according to embodiments hereof.

FIG. 3J is a schematic of a side cross-section of the distal portion of the delivery assembly of FIG. 3A with the transcatheter heart valve prosthesis fully deployed from the capsule of the delivery assembly and the delivery assembly retracted such that the dampening arms are removed from the transcatheter heart valve prosthesis according to embodiments hereof.

FIG. 4A is a schematic of a side cross-section of a distal portion of the delivery assembly of FIG. 2A in the delivery configuration, showing dampening arms including a pad according to embodiments hereof.

FIG. 4B is a schematic of a side cross-section of a distal portion of the delivery assembly of FIG. 2A with a capsule of the delivery system partially retracted according to embodiments hereof.

FIG. 4C shows a close up view of a mesh pad of the dampening arms of FIG. 4A according to embodiments hereof.

FIG. 4D shows a close up view of a mesh pad of the dampening arms of FIG. 4A according to embodiments hereof.

FIG. 4E is a schematic of a side cross-section of the distal portion of the delivery assembly of FIG. 4A with the transcatheter heart valve prosthesis deployed from the capsule of the delivery assembly according to embodiments hereof.

FIG. 4F is a schematic of a side cross-section of the distal portion of the delivery assembly of FIG. 4A with the transcatheter heart valve prosthesis fully deployed from the capsule of the delivery assembly and the delivery assembly retracted such that the dampening arms are removed from the transcatheter heart valve prosthesis according to embodiments hereof.

FIG. 4G is a flat, as-cut configuration of the transcatheter heart valve prosthesis and the spindle partially disposed within the capsule of the delivery device according to embodiments hereof.

### DETAILED DESCRIPTION

It should be understood that various embodiments disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single device or component for purposes of clarity, it should be understood that the techniques of this disclosure may be performed by a combination of devices or components associated with, for example, a delivery device. The following detailed description is merely exemplary in nature and is not intended to limit the invention of the application and uses of the invention. Furthermore, there is no intention to be bound by any expressed or implied theory presented in the preceding field of the invention, background, summary or the following detailed description.

As used in this specification, the singular forms “a”, “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%. It should be understood that use of the term “about” also includes the specifically recited number of value.

The terms “proximal” and “distal” when used herein with respect to a delivery system are used with reference to the clinician using the devices. Therefore, “proximal” and “proximally” mean in the direction toward the clinician, and “distal” and “distally” mean in the direction away from the clinician. The terms “proximal” and “distal” when used herein with respect to an implanted prosthetic heart valve device are used with reference to the direction of blood flow. Therefore, “proximal” and “proximally” mean upstream with respect to the direction of blood flow, and “distal” and “distally” mean downstream with respect to the direct of blood flow.

Further, numerical terms such as “first”, “second”, “third”, etc. used herein are not meant to be limiting such that use of the term “second” when referring to a part in the specification does not mean that there necessarily is a “first” of part in order to fall within the scope of the invention. Instead, such numbers are merely describing that the particular embodiment being described has a “first” part and a “second” part. The invention is instead defined by the claims, in which one or more of the numbered parts may be claimed.

Embodiments hereof relate to a delivery assembly including a spindle coupled to a middle member, the spindle having at least one dampening arm configured for use with a transcatheter heart valve prosthesis when loading the transcatheter heart valve prosthesis into a capsule for delivering the transcatheter heart valve prosthesis to a treatment site within a vasculature of a patient and deploying the transcatheter heart valve prosthesis at the treatment site. More particularly, the spindle is configured to prevent, or block, leaflets of the transcatheter heart valve prosthesis from protruding through cells or openings in a frame of the transcatheter heart valve prosthesis during loading of and deployment of the transcatheter heart valve prosthesis that may cause leaflet pinching and damage. The spindle includes at least one dampening arm or at least one dampening arm having a mesh pad such that when the transcatheter heart valve prosthesis is being loaded into the capsule, and/or is being deployed from the capsule, and/or is being recaptured into the capsule, the at least one dampening arm or dampening arm with a mesh pad, prevents the leaflets from protruding through an access cell of the frame of the transcatheter heart valve prosthesis, thereby minimizing potential damage to the leaflets during loading, deployment and/or recapture.

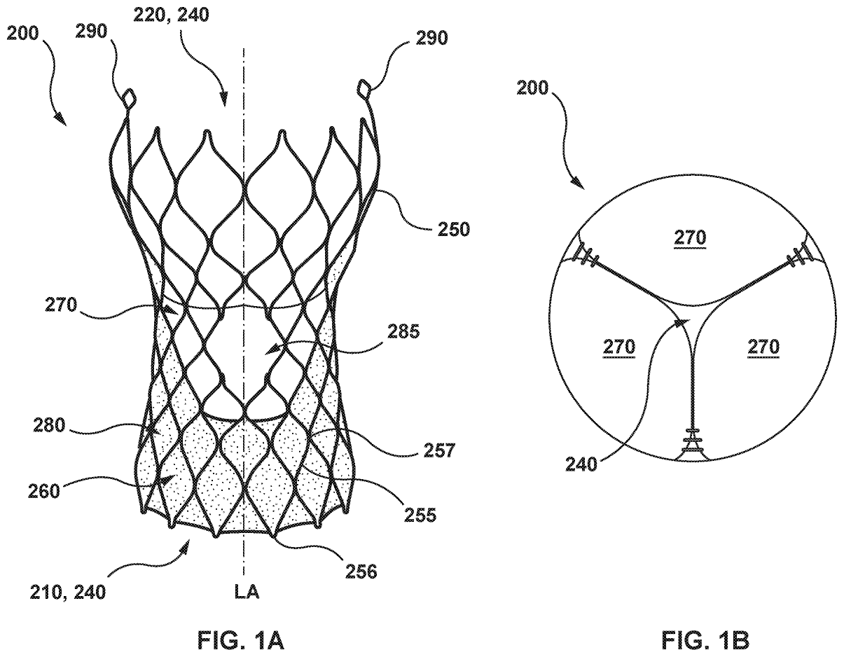

FIGS. 1A and 1B illustrate a side view and an outflow view, respectively, of a transcatheter heart valve prosthesis 200. The transcatheter heart valve prosthesis 200 includes a radially-expandable frame or stent 250 and a prosthetic valve 260. The frame 250 of the transcatheter heart valve prosthesis 200 supports the prosthetic valve 260 within the interior of the frame 250. In the example transcatheter heart valve prosthesis 200 shown in FIGS. 1A-1B, the frame 250 is self-expandable. However, this is not meant to be limiting, and the frame 250 can be balloon-expandable or mechanically expandable.

The prosthetic valve 260 includes at least one leaflet 270 disposed within and secured to the frame 250. In the embodiment shown in FIGS. 1A-1B, the prosthetic valve 260 includes exactly three leaflets 270, as shown in FIG. 1B. However, this is not meant to be limiting, as the prosthetic valve 260 may include more or fewer leaflets 270. The valve leaflets 270 open and close to regulate flow through the transcatheter heart valve prosthesis 200.

As shown in FIG. 1A, the transcatheter heart valve prosthesis 200 includes an inflow end 210 and an outflow end 220. The prosthetic leaflets 270 are attached to the frame 250 such that when pressure at the inflow end 210 exceeds pressure at the outflow end 220, the prosthetic leaflets 270 open to allow blood flow through the heart valve prosthesis 200 from the inflow end 210 to the outflow end 220. When the pressure at the outflow end 220 exceeds pressure at the inflow end 210, the prosthetic leaflets 270 close to prevent blood flow from the outflow end 220 to the inflow end 210.

The frame 250 of the transcatheter heart valve prosthesis 200 further includes a plurality of struts 255 that are arranged to form a plurality of side openings or cells 280 arranged circumferentially around a longitudinal axis LA of the transcatheter heart valve prosthesis 200 and longitudinally to form a tubular structure defining a central lumen 240 of the transcatheter heart valve prosthesis 200. The struts 255 are defined herein as the elongated wire segments of the frame 250. The struts 255 come together to form crowns 256 or nodes 257, as can be seen in FIG. 1A. The frame 250 is configured to secure the prosthetic valve 260 within the central lumen 240 of the frame 250 and to secure the transcatheter heart valve prosthesis 200 in place in the vasculature of the patient.

As noted above, the struts 255, crowns 256, and nodes 257 define the plurality of cells 280. In the example shown in FIG. 1A, the plurality of cells 280 may be diamond-shaped. In the example shown, the plurality of cells includes a plurality of first cells 280 and a plurality of access cells 285. In particular, the access cells 285 are larger than the first cells 280. In the embodiment shown, there are exactly three access cells 285. However, this is not meant to be limiting, as the frame 250 of the transcatheter heart valve prosthesis 200 can include more or fewer access cells 285. The access cells 285 each have an enlarged area relative or compared to the first cells 280, as can be seen in FIG. 1A. Further, the access cells 285 may be located in other locations than the locations shown in FIG. 1A.

The frame 250 may further include a plurality of tabs or paddles 290 for coupling the heart valve prosthesis 200 to a delivery system. In the embodiment shown, there are three paddles 290, with each aligned with a corresponding commissure of the prosthetic valve 260 (only two are shown in FIG. 1A due to the view). Further, although shown as symmetrically disposed around the circumference of the frame 250, the tabs need not be symmetrically disposed, as described, for example, In U.S. Published Application Publication No. 2022/0175524, which is incorporated by reference herein in its entirety. Further, in other embodiments, more or fewer paddles 290 may be utilized.

As explained above, a delivery assembly may be used to deliver the transcatheter heart valve prosthesis 200 in a crimped configuration and to deploy the transcatheter heart valve prosthesis 200 in an expanded configuration at the treatment site within the vasculature. When the heart valve prosthesis 200 is loaded into the delivery assembly or recaptured into the delivery assembly, the frame 250 is radially compressed or crimped to the crimped configuration. As the frame 250 is compressed, the first cells 280 and the access cells 285 also reduce in size, potentially trapping or pinching the one or more of the leaflets 270 between the struts 255 defining the first cells 280 and the access cells 285. Further, during the deployment process, expansion of the frame 250 from the crimped configuration to the expanded configuration causes the area of the cells 280, 285 to increase in size. In some instances, one or more of the leaflets 270 of the prosthetic valve 260 may protrude through one of the cells 280, 285 during the expansion process, which may cause the leaflets 270 to get pinched, or stuck between the struts 255 of the frame 250 and sustain damage such as tearing. Leaflet protrusion becomes increasingly likely as the area of the cells increase. Therefore, it is more likely to occur at the access cells 285 than the first cells 280 of the frame 250. Accordingly, embodiments of a delivery assembly of the present invention minimize the risk of leaflet protrusion, as described in further detail below.

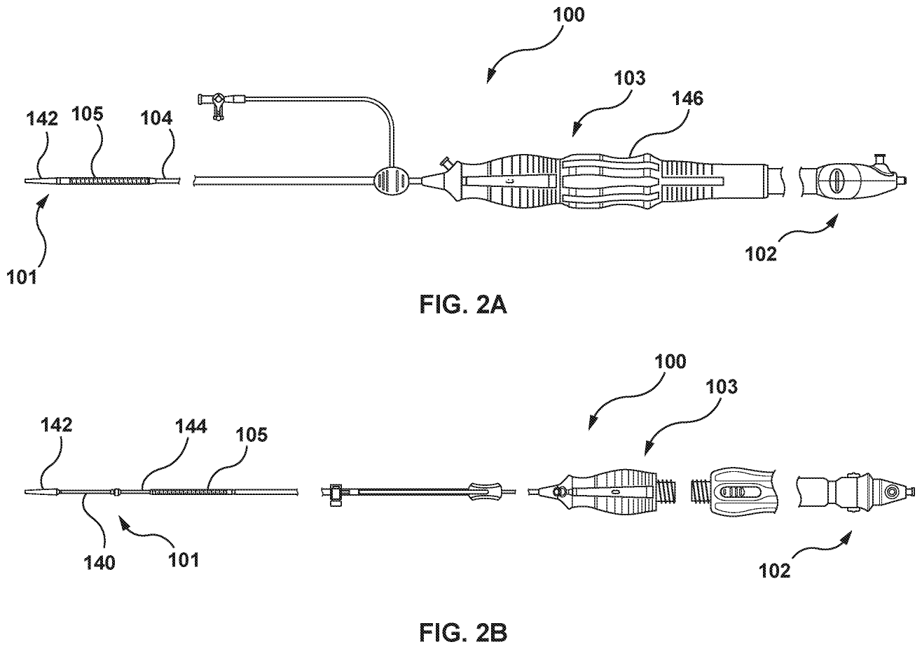

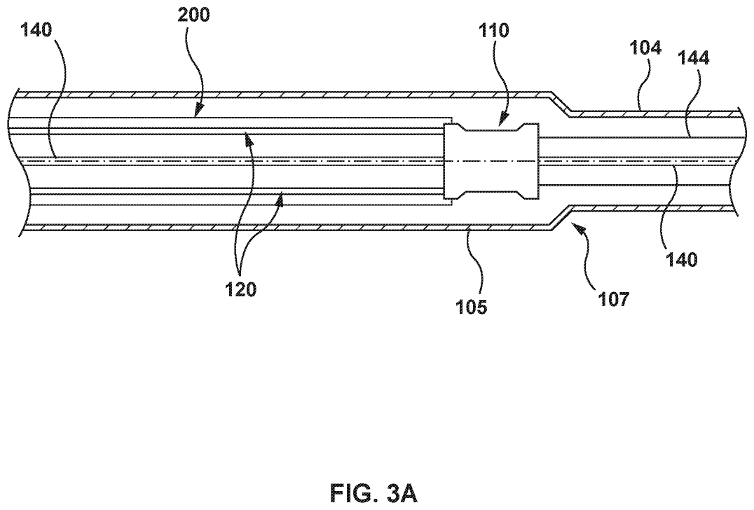

FIGS. 2A-2B show schematic side view of a delivery assembly 100 for delivering and deploying a transcatheter heart valve prosthesis according to embodiments hereof. One skilled in the art will realize that FIGS. 2A-2B illustrate one example of a delivery assembly and that components illustrated in FIGS. 2A-2B may be removed and/or additional components may be added. The delivery assembly 100 extend from a proximal end 102 to a distal end 101. The delivery assembly 100 includes, inter alia, a handle 103 at the proximal end 102, an outer shaft 104 extending distally from the handle 103, an inner shaft 140 disposed within the outer shaft 104, and a middle member 144 disposed between the inner shaft 140 and the outer shaft 104. The handle 103 enables a clinician to manipulate a distal portion of the delivery assembly 100 and includes actuators for moving parts of the delivery system relative to other parts. In the delivery system 100, the outer shaft 104 is coupled to an actuator 146 of the handle 103 for moving the outer shaft 104 (and the capsule noted below) relative to the inner shaft 140, as shown in FIG. 2B. A distal portion of the outer shaft 104, referred to as a capsule 105, is configured to surround a transcatheter heart valve prosthesis during delivery to the treatment site, e.g., a native heart valve, and the capsule 105 is retracted from the transcatheter heart valve prosthesis to expose the transcatheter heart valve prosthesis such that it self-expands. The inner member 140 is coupled to the handle 103 and movement of the handle 103 translates to movement of the inner member 140 and a distal tip 142 coupled to a distal end of the inner member 140. The inner member 140 and the distal tip 142 may also be translated relative to the outer shaft 104 and the handle 103 via a tip retractor. In the embodiment shown, the middle member 144 is disposed between the inner member 140 and the outer shaft 104, and the middle member 144 may include a retainer or spindle 110 attached to a distal portion thereof for receiving the paddles 290 of the transcatheter heart valve prosthesis 200. As known to those skilled in the art, when the delivery assembly 100 is in position such that the transcatheter heart valve prosthesis 200 is at the desired position at the treatment site in the patient's vasculature, the actuator 146 is actuated to move the capsule 105 relative to the inner shaft 140 and the transcatheter heart valve prosthesis 200 disposed between the inner shaft 140 and the capsule 105, thereby enabling the transcatheter heart valve prosthesis 200 to deploy via self-expansion at the treatment site, as shown in FIG. 2B (without showing the transcatheter heart valve prosthesis 200).

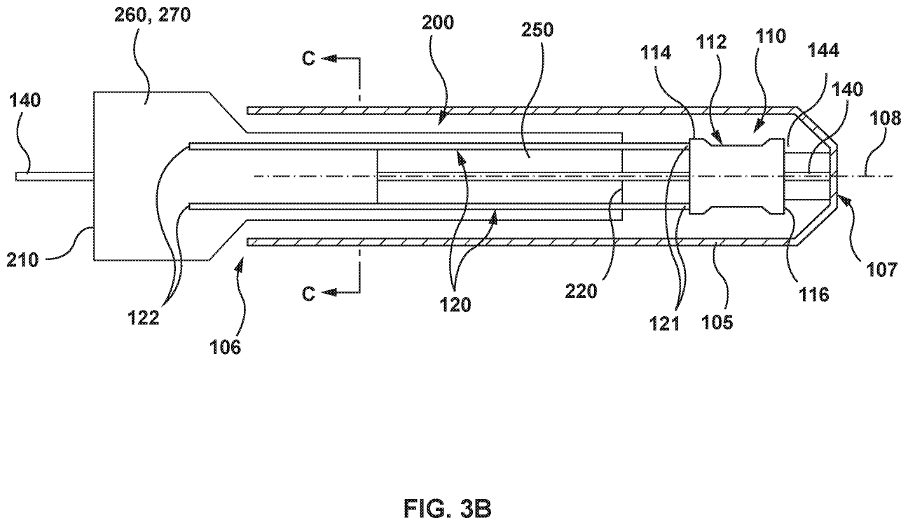

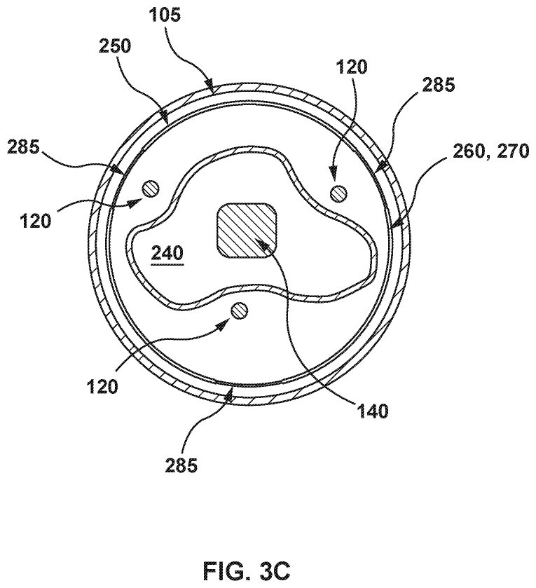

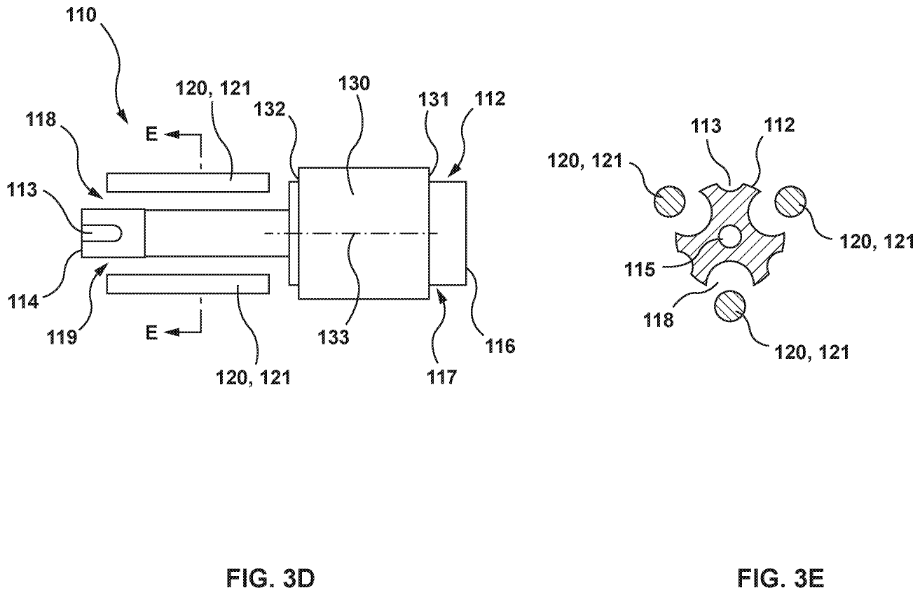

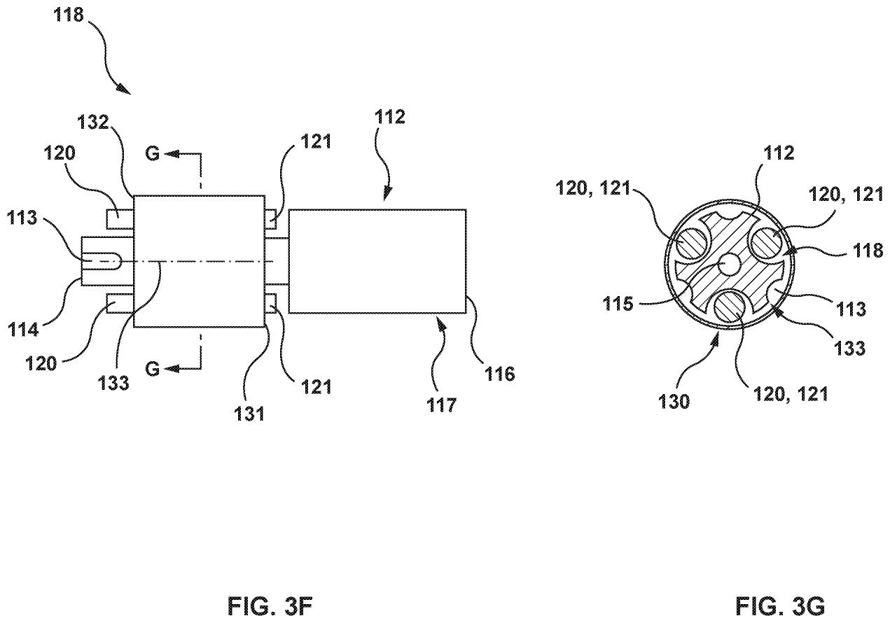

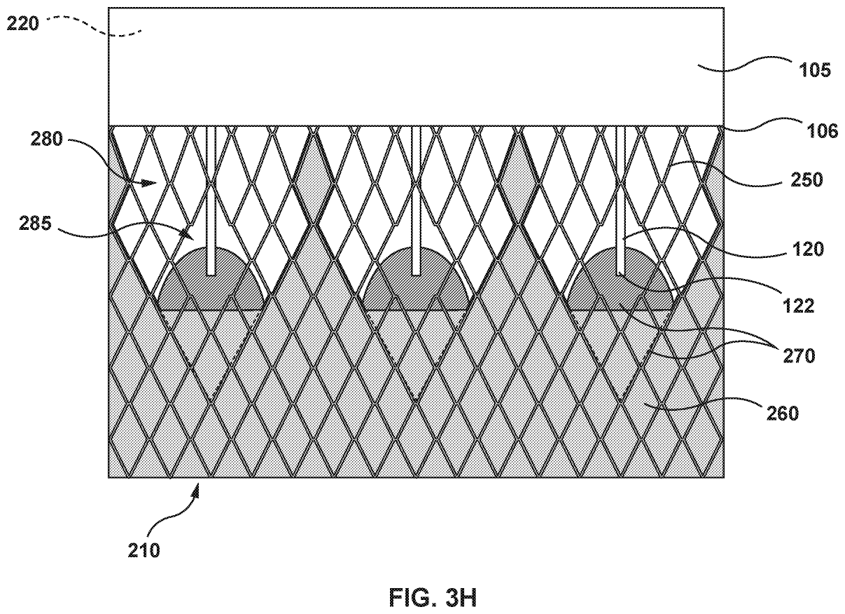

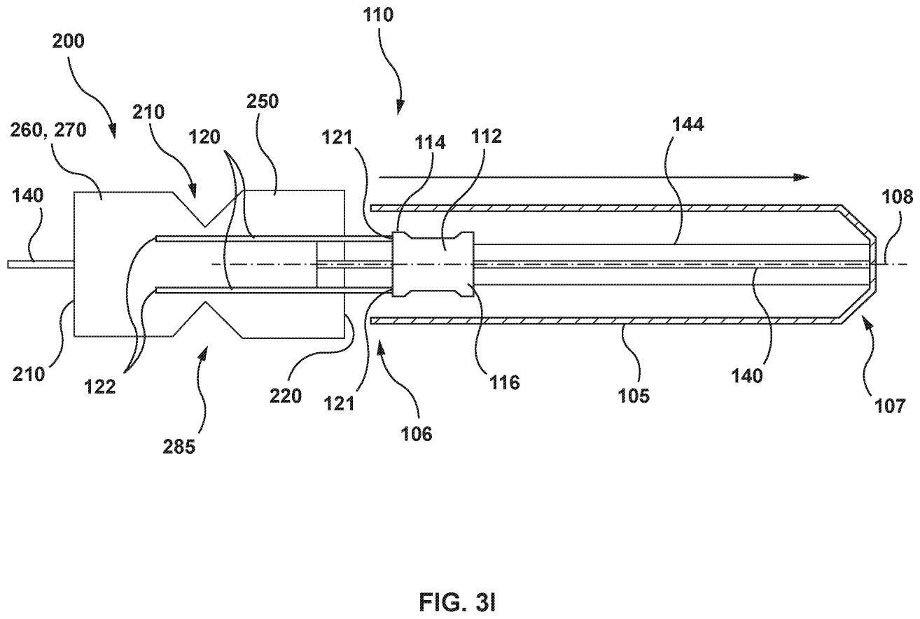

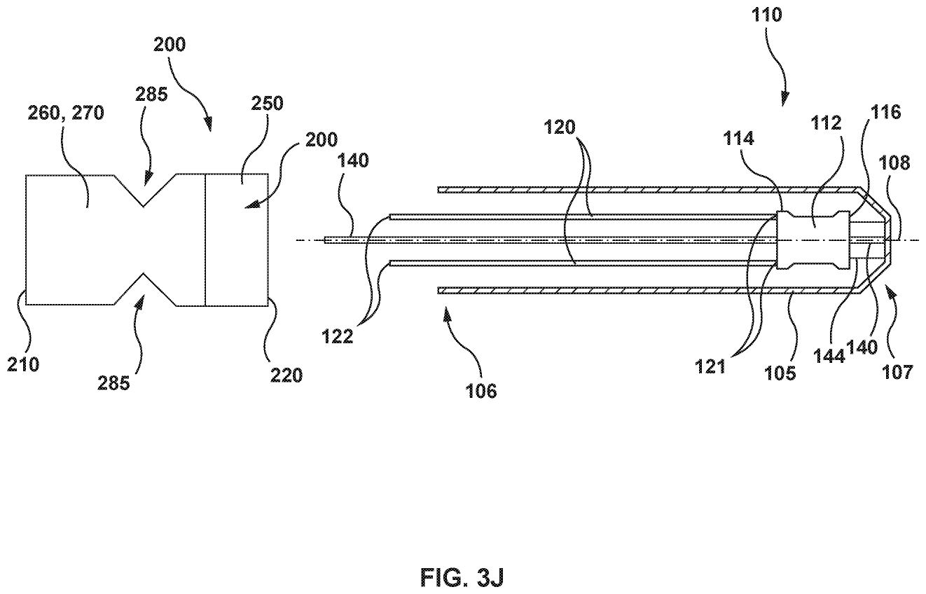

As explained above, in the delivery configuration, the capsule 105 of the delivery assembly 100 contains the transcatheter heart valve prosthesis 200 while the delivery assembly 100 is advanced to a desired treatment site. FIGS. 3A-3G show an embodiment of a delivery system 100 including a mechanism to minimize leaflet protrusion, according to embodiments hereof. One skilled in the art will realize that FIGS. 3A-3G illustrate one example of a delivery system 100 and that components illustrated in FIGS. 3A-3G may be removed and/or additional components may be added. As shown, the middle member 140 includes a spindle, as will be described in more detail below.

As shown in FIGS. 3A and 3B, the capsule 105 of the delivery system 100 is a longitudinal tube having an open distal end 106, a proximal end 107, and a central lumen 108 extending from the proximal end 107 to the distal end 106. The proximal end 107 of the capsule 105 is coupled to the outer shaft 104. The inner shaft 140 extends through the central lumen 108 of the capsule 105, as shown in FIG. 3A. The middle member 144 is disposed over the inner shaft 140 between the inner shaft 140 and the outer shaft 104. The central lumen 108 of the capsule 105 is sized and shaped to contain the transcatheter heart valve prosthesis 200 therein in the crimped configuration.

As shown in FIGS. 3A and 3B, the inner shaft 140 extends through the central lumen 108 of the capsule 105 and is coupled at its distal end to a distal tip (not shown), as known to those skilled in the art. The spindle 110 is coupled to a distal end of the middle member 144. In other embodiments, the spindle 110 may be coupled to other portions of the delivery system 100, such as the inner shaft 140. The spindle 110 generally includes a spindle body 112 and at least one dampening arm 120 coupled to the spindle body 112. The spindle body 112 includes a distal end 114 and a proximal end 116. The spindle body 112 is coupled to the middle member 140. More particularly, the spindle body 112 is coupled to a distal end of the middle member 144, and the inner shaft 140 extends through a central opening or passageway 115 of the spindle body 112.

In the embodiment shown, the delivery system 100 includes three dampening arms 120 corresponding to the three leaflets 270 and the three access cells 285 of the frame 250. However, this is not meant to be limiting, and more or fewer dampening arms 120 may be used depending on the situation. Each dampening arm 120 includes a first end or proximal end 121 coupled to the spindle body 112 and a second end 122 extending distally from the spindle body 112, as shown in FIGS. 3A and 3B. The dampening arms 120 may be secured to the spindle body 112 by a collar 130, as described in detail below with respect to FIGS. 3D-3G.

As stated previously, the spindle 110 including the dampening arms 120 of the delivery system 100 is configured to prevent leaflets 270 of the prosthetic valve 260 from protruding through the cells 280, 285 of the frame 250 during loading, recapture and/or deployment of the transcatheter heart valve prosthesis 200. More specifically, the dampening arms 120 are configured to prevent the leaflets 270 from protruding through the enlarged access cells 285 of the frame 250 of the transcatheter heart valve prosthesis 200, according to embodiments hereof. Accordingly, as show in FIGS. 3A and 3B, the transcatheter heart valve prosthesis 200 is loaded into the capsule 105 of the delivery system 100 such that the dampening arms 120 of the spindle 110 extend between the frame 250 and the prosthetic valve 260 of the transcatheter heart valve prosthesis 200. FIG. 3A shows a cross-sectional view of the capsule 105 with the transcatheter heart valve prosthesis 200 loaded therein, and FIG. 3B shows a cross-section view of the capsule 105 partially retracted therefrom, either during loading of the transcatheter heart valve prosthesis 200 into the capsule 105 or deployment of the transcatheter heart valve prosthesis 200 from the capsule 105.

As can be seen in FIGS. 3A-3C and 3H, each dampening arm 120 extends from the spindle 110, into the outflow end 220 the frame 250, and between a corresponding one of the leaflets 270 and the frame 250. In other words, the dampening arms 120 are disposed radially inward from the frame 250 of the heart valve prosthesis 200, but are disposed radially outward from the prosthetic leaflets 270 of the heart valve prosthesis 200. Due to the location of the leaflets 270 and the access cells 285, the distal end 122 of each dampening arm 120 approximately longitudinally bisects the corresponding access cell 285, as best shown in FIG. 3H. Each dampening arm 120 presses radially inwardly on the corresponding leaflet 270, thereby preventing or minimizing the likelihood that the leaflets 270 protrude through the access cells 285 during loading, recapture, and/or deployment of the transcatheter heart valve prosthesis 200 into/from the capsule 105.

In some embodiments, the heart valve prosthesis 200 is crimped and loaded into capsule 105 using a funnel (not shown) at the geographic location where the heart valve prosthesis 200 is going to be implanted into a patient (e.g., a hospital). Inserting the dampening arms 120 into the heart valve prosthesis 200 as the heart valve prosthesis is being loading into the delivery system 100 may be difficult, and may cause damage to the leaflets 270. Therefore, in an embodiment, the dampening arms 120 may come pre-assembled within the transcatheter heart valve prosthesis 200 prior to loading the transcatheter heart valve prosthesis 200 within the delivery system 100, or may be inserted into the transcatheter heart valve prosthesis 200 prior to the dampening arms 120 being coupled to the spindle body 112. In such an embodiment, either at the manufacturing site or prior to the loading process at the geographic treatment location, the second ends 122 of the dampening arms 120 are advanced through the outflow end 220 of the heart valve prosthesis 200 and between the leaflets 270 and the frame 250. The first ends 121 of the dampening arms 120 extend out of the outflow end 220 of the heart valve prosthesis 200. The first ends 121 of the dampening arms 120 are then coupled to the spindle body 112.

FIGS. 3D-3I show various schematic views of an embodiment of the spindle 110 which shows a particular embodiment for coupling the dampening arms 120 to the spindle body 112. Those skilled in the art will recognize that FIGS. 3D-3I shown one particular embodiment of the spindle 110 and a device/method for coupling the dampening arms 120 to the spindle body 112, and is not meant to be limiting. In the embodiment shown, the spindle 110 includes the spindle body 112 and a collar 130 for coupling the dampening arms 120 to the spindle body 112. FIG. 3D shows a schematic side view of the spindle 110, and FIG. 3E shows a cross-section taken at line E-E in FIG. 3D. FIGS. 3D and 3E shown the spindle body 112 with the three dampening arms 120 uncoupled to the spindle body 112 and the collar in a retracted position.

The spindle body 112 includes a proximal end 116 and a distal end 114. The central passageway 115 extends from the proximal end 116 to the distal end 114 and is configured to receive the inner shaft 140 therethrough. The central passageway 115 may also house a distal end of the middle member 144 if the delivery system 100 includes such a middle member 144. The spindle body 112 includes a proximal portion 117 and a distal portion 119. The distal portion 119 of the spindle body 112 includes a plurality of cavities 118, each of which is sized and shaped to receive a corresponding one of the plurality of dampening arms 120 therein, as best seen in FIG. 3E. Thus, in the embodiment shown, each cavity is a substantially semi-circular shaped channel that extends into the spindle body 112. In the embodiment shown, the spindle body 112 includes exactly three cavities 118 equidistantly spaced apart from one another around a circumference of the spindle body 112. The distal portion 119 of the spindle body 112 may also include at least one paddle pocket 113 configured to receive a paddle 290 of the heart valve prosthesis 200. In the embodiment shown, there are three paddle pockets 113, with each paddle pocket 113 sized and shaped to receive a corresponding one of the three paddles 290 of the heart valve prosthesis 200.

FIG. 3D shows a side view of the spindle body 112, the dampening arms 120 and the collar 130 of the spindle 110. In an embodiment, the longitudinal length of the spindle body 112 may be in the range of about 7 mm to about 8 mm. The longitudinal length of each cavity 118 may be in the range of about 4 mm to about 5 mm. As can be seen in FIG. 3C, each cavity 118 extends proximally from the distal end 114 of the spindle body 112 and terminates distal to the proximal end 116 of the spindle body 112.

The locking collar 130 (herein also referred to as collar 130) of the spindle 110 is a substantially tubular-shaped element that includes a first end 131, a second end 132, and a collar lumen 133 extending from the first end 131 to the second end 132. The locking collar 130 is configured to secure or lock the dampening arms 120 within the cavities 118 of the spindle body 112. Prior to loading the dampening arms 120 within the cavities 118 of the spindle body 112, the collar 130 is disposed over the proximal portion 117 of the spindle body 112, as shown in FIG. 3D. Thus, the collar lumen 133 of the locking collar 130 is sized and shaped to receive the spindle body 112. Further, the second end 132 of the collar 130 is disposed distal to the first end of the collar 130, and the second end 132 of the collar 130 terminates proximal to the cavities 118 of the distal portion 119 of the spindle body 112 prior to coupling the dampening arms 120 to the spindle body 112, as shown in FIG. 3D.

FIG. 3F shows a schematic side view of the spindle body 112 with the dampening arms 120 disposed within the cavities 118 and the collar 130 translated distally to cover the dampening arms 120. FIG. 3G shows a cross-sectional view of the spindle 110 taken at line G-G of FIG. 3F. As can be seen, with each dampening arm 120 disposed in a respective cavity 118 of the spindle body 112, the collar 130 is translated distally to couple the dampening arms 120 to the spindle body 112. As explained above, in some embodiments, the second ends 122 of the dampening arms 120 are disposed within frame 250 of the heart valve prosthesis 200 prior to crimping and loading the heart valve prosthesis 200 into the capsule 205. Thus, during the crimping and loading process, the first ends 121 of the dampening arms 120 may be coupled to the spindle body 112 by placing the first ends 121 of the dampening arms 120 in the cavities 118 and sliding the collar 130 distally to cover the cavities 118 with the dampening arms 120 disposed therein. The spindle body 112 and the collar 130 may include a mechanism for locking the collar 130 in the distal position, such as, for example and not by way of limitation, a snap fit mechanism, a bump or similar feature in the spindle body 112 that the collar 130 must be pushed over, or a similar locking feature.

As stated previously, FIG. 3A shows the transcatheter heart valve prosthesis 200 loaded within the capsule 105 of the delivery system 100 in the crimped or compressed configuration, with the first ends 121 of dampening arms 120 coupled to the spindle body and the second ends 122 of the dampening arms disposed between the leaflets 270 and the frame 250 of the heart valve prosthesis 200. Upon delivery of the delivery system 100 to a treatment site within the vasculature of a patient, such as a native aortic valve, the transcatheter heart valve prosthesis 200 is deployed from the delivery system 100 and self-expands within the native valve of the patient.

To transition the delivery system 100 from a delivery configuration to a deployed configuration, the capsule 105 is retracted proximally in order to release, or uncover, the transcatheter heart valve prosthesis 200. In other words, as the open distal end 106 of the capsule 105 is retracted proximally, the inner shaft 140, the middle member 140, the spindle 110, the dampening arms 120, and the transcatheter heart valve prosthesis 200 remain in place such that they exit the central lumen 108 of the capsule 105 through the distal end 106. As the capsule 105 is retracted proximally, the inflow end 210 of the transcatheter heart valve prosthesis 200 may begin to self-expand in the vasculature, as it is no longer enclosed by the capsule 105. As the capsule 105 translates proximally and uncovers or releases the transcatheter heart valve prosthesis 200, the dampening arms 120 of the spindle extension 110 remain disposed between the interior surface of the frame 250 and the leaflets 270 of the prosthesis 200 to prevent leaflet pinching and/or damage while the frame 250 expands. More particularly, each second end 122 of each dampening arm 120 remains radially aligned with each access cell 285 of the frame 250 and prevents or inhibits each leaflet 270 from moving radially outward such that the leaflets 270 are prevented or inhibited from extending or protruding through the access cells 285, as shown in FIG. 3B. With the dampening arms 120 in place during retraction of the capsule 105, if recapture of the heart valve prosthesis 200 is needed after partial expansion of the heart valve prosthesis 200 (as shown in FIG. 3B), such as for repositioning of the heart valve prosthesis 200, the dampening arms 120 prevent or inhibit leaflet pinching while the frame 250 is re-compressed into the capsule 105.

Once the delivery system 100 is in the final position for deploying the heart valve prosthesis 200, the capsule 105 is fully retracted proximal of the transcatheter heart valve prosthesis 200 such that the entire transcatheter heart valve prosthesis 200 is released from the capsule 105 and is fully expanded at the treatment site within the vasculature, as shown in FIG. 3I. The delivery system 100 can then be retracted by moving the handle 103 proximally such that the inner shaft 140, the middle member 144, the distal tip 142, the outer shaft 104, the capsule 105, and spindle 110 including the dampening arms 120 coupled to the spindle body 112 are retracted proximally with the handle 103 to remove the dampening arms 120 from within the heart valve prosthesis 200, as shown in FIG. 3J. The delivery system 100 can then be removed from the vasculature of the patient.

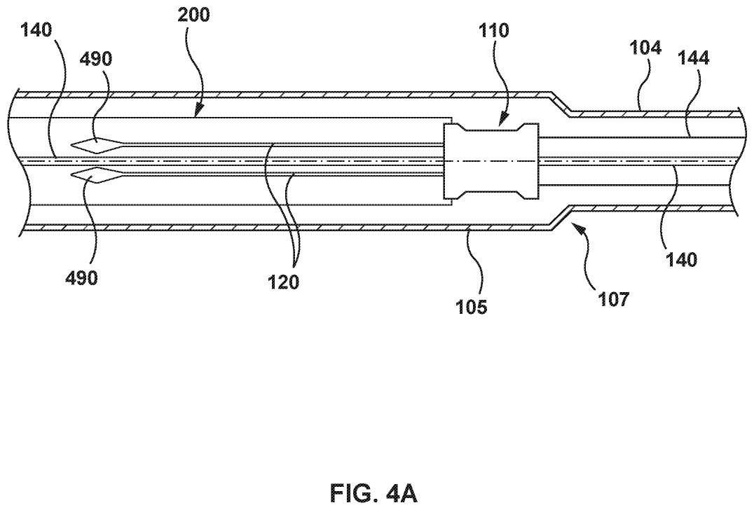

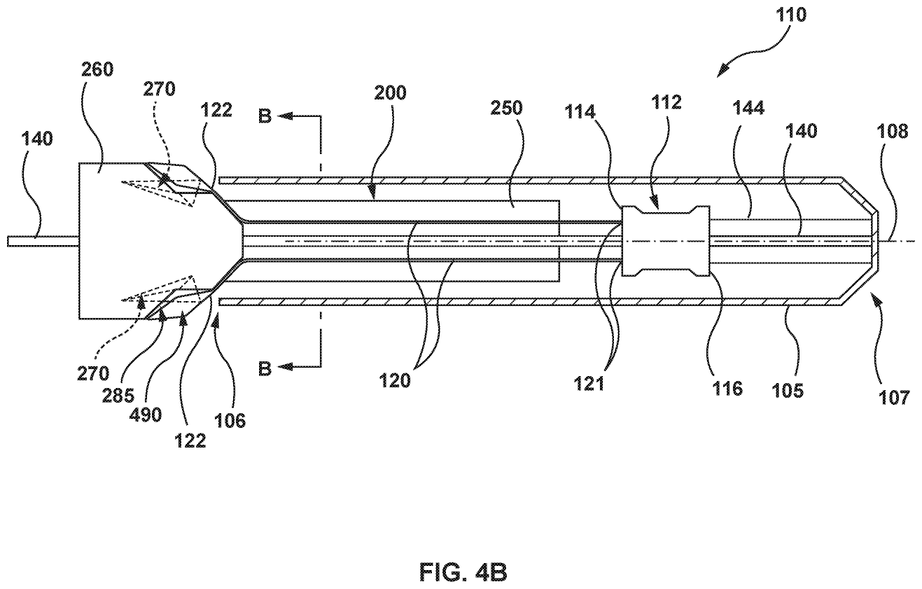

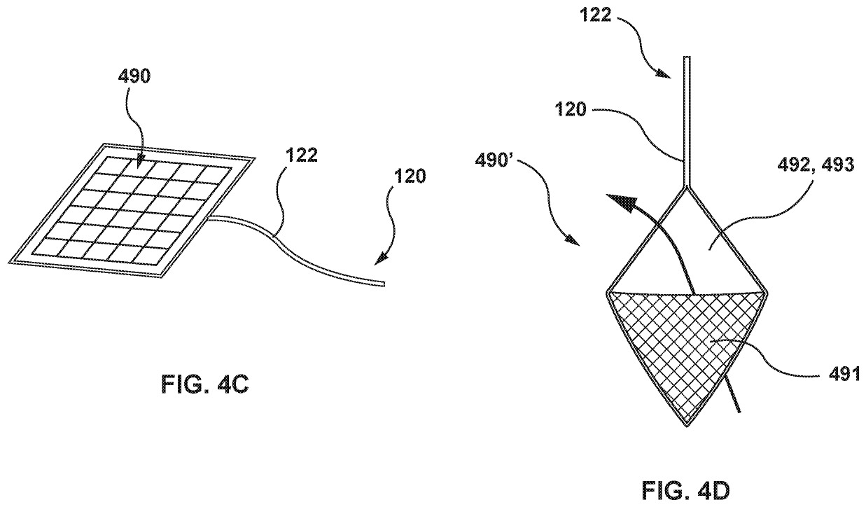

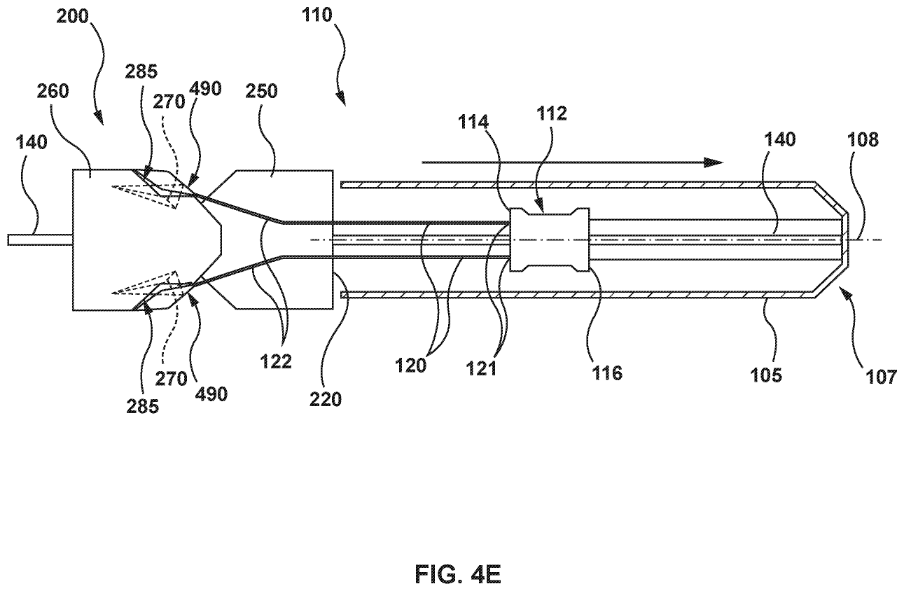

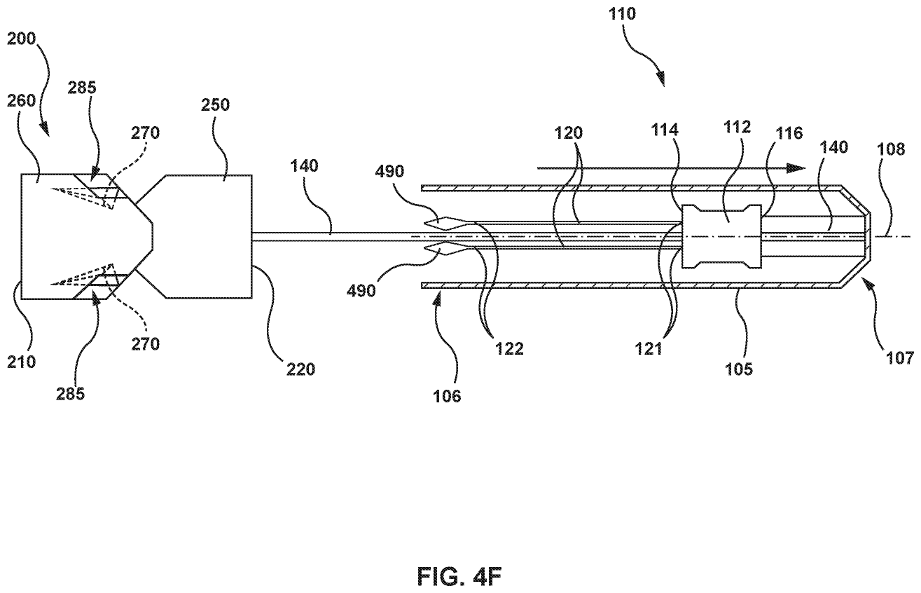

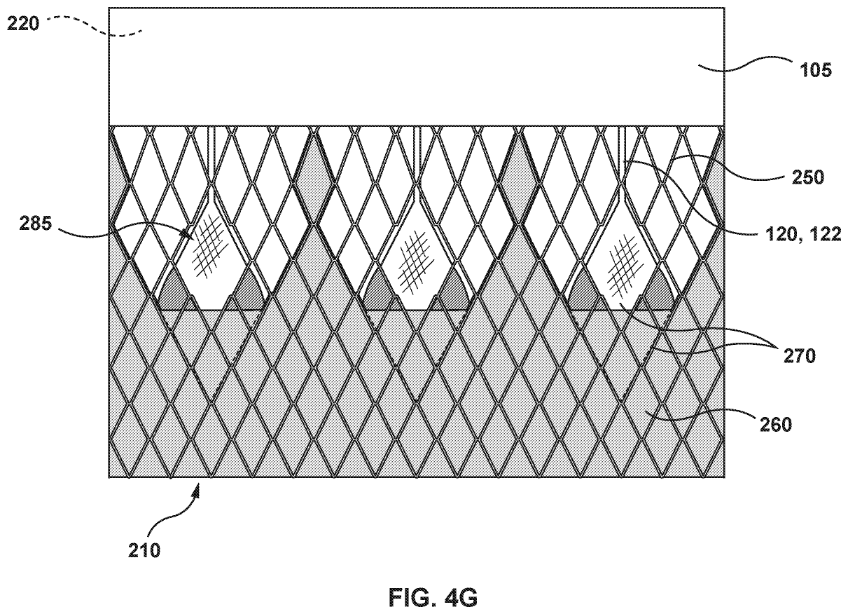

FIGS. 4A-4G show another embodiment of a spindle 110 including dampening arms 120 according to embodiments hereof. As shown and described with respect to FIGS. 4A-4F, the dampening arms 120 may each optionally include a pad 490 coupled to the respective second ends 122 thereof. The pad 490 provides a larger surface area for each dampening arm 120 to prevent or inhibit the leaflets 270 of the prosthesis 200 from protruding through the access cells 285 of the frame 250 during loading, recapture and/or deployment of the transcatheter heart valve prosthesis 200.

As shown in FIGS. 4A and 4B, the delivery system 200 is the same as the delivery system 200 described above with respect to FIGS. 2A-2B and FIGS. 3A-3I, except for the pads 490 at the second ends 122 of each dampening arm 120. Therefore, the details of the delivery system are not repeated with respect to FIGS. 4A-4G. In particular, and not by way of limitation, the spindle body 112, locking collar 130, cavities 118, and paddle pockets 113 described above with respect to FIGS. 3D-3G are incorporated into the embodiment of FIGS. 4A-4G. Similarly, any details with respect to FIGS. 2A-2B and FIGS. 3A-3I not specifically distinguished with respect to FIGS. 4A-4G are incorporated into the embodiment of FIGS. 4A-4G.

As described above, the distal end 122 of each dampening arm 120 may include a pad 490. In an embodiment, the pad 490 may be substantially diamond-shaped. In an embodiment, the pad 490 may be substantially the same size and shape as an access cell 285 of the frame 250, but this is not meant to be limiting. In an embodiment, the pad 490 may a mesh pad to enable compression thereof when crimped inside the capsule 105. Further, a pad 490 form of mesh may enable blood flow through the openings in the mesh. In another embodiment, shown in FIG. 4D, the pads 490′ material (such as a mesh material) covering a distal portion 491 of the mesh pad 490′ and a gap 493 at a proximal portion 492 of the mesh pad 490′. In the embodiment shown, the distal portion 491 of the diamond-shaped pad 490′ is substantially triangular shaped and is covered with the covering material. The proximal portion 492 of the pad 490′ is substantially triangular shaped and is not covered by material, thus forming an opening or gap 493 in the proximal portion 492. The proximal portion 492 of the pad 490′ is coupled to the second end 122 of the dampening arm 120. The gap 493 of the proximal portion 492 of the pad 490′ allows for blood flow during the delivery procedure of the prosthesis 200, as indicated by the arrow in FIG. 4D. However, this is not meant to be limiting, as the pad 490′ may be a different shape. Thus, the pad 490 includes a material portion and a gap that enables blood flow therethrough.

The pad 490, 490′ can be made of an atraumatic soft polymer, such as, but not limited to, polytetrafluoroethylene (PTFE). The durometer of the material of the pad may be in the range of 45 to 60 Shore D. The material may be a low friction material to avoid damaging the tissue of the prosthetic valve 260.

The pads 490 of the dampening arms 120 extend between the leaflets 270 of the prosthetic valve 260 and the frame 250 of the heart valve prosthesis 200, as described above. However, in some embodiments, the each dampening arm 120 may extend within the frame 250 until the dampening arm 120 reaches the corresponding access cell 285. The pad 490 at the distal end 122 of the dampening arm 120 may then extend outside of the frame 250 such that a portion of the pad 490 contacts outer surface of the frame 150, as best shown in FIG. 4G.

Similar to the embodiment described above, FIG. 4A shows the transcatheter heart valve prosthesis 200 loaded within the capsule 105 of the delivery system 100 in the crimped or compressed configuration, with the first ends 121 of dampening arms 120 coupled to the spindle body 112 and the pads 490 at the second ends 122 of the dampening arms 120 disposed outside of the leaflets 270 of the heart valve prosthesis 200. Upon delivery of the delivery system 100 to a treatment site within the vasculature of a patient, such as a native aortic valve, the transcatheter heart valve prosthesis 200 is deployed from the delivery system 100 and self-expands within the native valve of the patient.

To transition the delivery system 100 from a delivery configuration to a deployed configuration, the capsule 105 is retracted proximally in order to release, or uncover, the transcatheter heart valve prosthesis 200. In other words, as the open distal end 106 of the capsule 105 is retracted proximally, the inner shaft 140, the middle member 140, the spindle 110, the dampening arms 120, and the transcatheter heart valve prosthesis 200 remain in place such that they exit the central lumen 108 of the capsule 105 through the distal end 106. As the capsule 105 is retracted proximally, the inflow end 210 of the transcatheter heart valve prosthesis 200 may begin to self-expand in the vasculature, as it is no longer enclosed by the capsule 105. As the capsule 105 translates proximally and uncovers or releases the transcatheter heart valve prosthesis 200, the dampening arms 120 of the spindle 110 remain disposed within the interior surface of the frame 250, and the pads 490 remained disposed radially outside of the leaflets 270 of the prosthesis 200 to prevent leaflet pinching and/or damage while the frame 250 expands. More particularly, each pad 490 of each dampening arm 120 remains radially aligned with each access cell 285 of the frame 250 and prevents or inhibits each leaflet 270 from moving radially outward such that the leaflets 270 are prevented or inhibited from extending or protruding through the access cells 285, as shown in FIG. 4B. With the dampening arms 120 and pads 490 in place during retraction of the capsule 105, if recapture of the heart valve prosthesis 200 is needed after partial expansion of the heart valve prosthesis 200 (as shown in FIG. 4B), such as for repositioning of the heart valve prosthesis 200, the pads 490 prevent or inhibit leaflet pinching while the frame 250 is re-compressed into the capsule 105.

Once the delivery system 100 is in the final position for deploying the heart valve prosthesis 200, the capsule 105 is fully retracted proximal of the transcatheter heart valve prosthesis 200 such that the entire transcatheter heart valve prosthesis 200 is released from the capsule 105 and is fully expanded at the treatment site within the vasculature, as shown in FIG. 4E. As can be seen in FIG. 4E, the dampening arms 120 remain within the frame 250 and the pads 490 remain outside of the leaflets 270. The delivery system 100 can then be retracted by moving the handle 103 proximally such that the inner shaft 140, the middle member 144, the distal tip 142, the outer shaft 104, the capsule 105, and spindle 110 including the dampening arms 120 and pads 490 coupled to the spindle body 112 are retracted proximally with the handle 103 to remove the dampening arms 120 from within the heart valve prosthesis 200, as shown in FIG. 4F. The delivery system 100 can then be removed from the vasculature of the patient.

It should be understood that various embodiments disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single device or component for purposes of clarity, it should be understood that the techniques of this disclosure may be performed by a combination of devices or components.

The following examples are illustrative of the techniques described therein.

Example 1. A delivery assembly for percutaneously delivering a transcatheter heart valve prosthesis into a vasculature of a patient, the delivery assembly comprising a capsule configured to constrain the transcatheter heart valve prosthesis therein; and a spindle including: a spindle body coupled to a shaft disposed within the capsule; and a dampening arm having a first end and a second end, the first end being coupled to the spindle body and the second end of the dampening arm being configured to extend between an interior surface of a frame and a leaflet of the transcatheter heart valve prosthesis to minimize leaflet protrusion through cells of the frame of the transcatheter heart valve prosthesis.

Example 2. The delivery assembly of Example 1, further comprising a transcatheter heart valve prosthesis, the transcatheter heart valve prosthesis including a frame and a valve structure coupled to the frame, wherein the valve structure includes a plurality of leaflets, and wherein with the transcatheter heart valve prosthesis radially compressed within the capsule, the dampening arm extends within the frame with the second end of the dampening arm disposed radially outside of a leaflet of the plurality of leaflets.

Example 3. The delivery assembly of Example 2, wherein the frame of the transcatheter heart valve prosthesis includes access cells disposed adjacent the plurality of leaflets of the valve structure, wherein the access cells are larger than other cells of the frame.

Example 4. The delivery assembly of Example 2 or Example 3, wherein the transcatheter heart valve prosthesis includes exactly three leaflets and exactly three access cells, and the spindle includes exactly three dampening arms.

Example 5. The delivery assembly of any one of Examples 1 through 4, wherein the spindle body further includes a cavity configured to receive the first end of the dampening arm.

Example 6. The delivery assembly of Example 5, wherein the spindle body includes exactly three cavities equidistantly spaced apart from one another around a circumference of the spindle body.

Example 7. The delivery assembly of Example 5 or Example 6, wherein the cavity is disposed at a distal portion of the spindle body such that a proximal portion of the spindle body does not contain the cavity.

Example 8. The delivery assembly of any one of Examples 5 through 7, wherein the spindle further comprises a locking collar configured to secure the first end of the dampening arm to the spindle body.

Example 9. The delivery assembly of Example 8, wherein spindle includes a first configuration wherein the locking collar is longitudinally spaced from the cavity such that the cavity is open to receive the first end of the damping arm and the spindle includes a second configuration wherein the locking collar surrounds the cavity with the first end of the dampening arm disposed in the cavity to couple the dampening arm to the spindle body.

Example 10. The delivery assembly of any one of Examples 1 through 9, wherein the dampening arm further includes a pad coupled to the second end thereof.

Example 11. The delivery assembly of Example 10, wherein the pad is substantially diamond-shaped.

Example 12. The delivery assembly of Example 10 or 11, wherein the pad is substantially the same size as the access cell of the transcatheter heart valve prosthesis.

Example 13. The delivery assembly of Example 12, wherein the pad comprises a mesh material.

Example 14. The delivery assembly of Example 12, wherein a first portion of the pad includes a covering material and a second portion of the pad is open to enable blood flow therethrough.

Example 15. A method of delivering and deploying a transcatheter heart valve prosthesis to a treatment site within a vasculature of a patient, the method comprising advancing a delivery device to the treatment site with the transcatheter heart valve prosthesis radially compressed within a capsule of the delivery device, the transcatheter heart valve prosthesis including a frame and a valve structure coupled to the frame, wherein the valve structure includes a plurality of leaflets, wherein the delivery device includes a spindle coupled to a shaft of the delivery device disposed within the capsule, and a dampening arm extending from the spindle with a distal end of the dampening arm disposed between an interior surface of the frame and a leaflet of the plurality of leaflets of the transcatheter heart valve prosthesis; proximally retracting the capsule to partially release the transcatheter heart valve prosthesis from the capsule, wherein the spindle of the delivery device do not move relative to the transcatheter heart valve prosthesis such that the distal end of the dampening arm remains disposed between the interior surface of the frame and the leaflet; further proximally retracting the capsule further to fully deploy the transcatheter heart valve prosthesis; and proximally retracting the shaft and the spindle from the transcatheter heart valve prosthesis such that the dampening arm retracts from the transcatheter heart valve prosthesis.

Example 16. The method of Example 15, further comprising removing the delivery device from the vasculature of the patient.

Example 17. The method of Example 15 or Example 16, further comprising: after proximally retracting the capsule to partially release the transcatheter heart valve prosthesis from the capsule and before further proximally retracting the capsule to fully release the transcatheter heart valve prosthesis, distally advancing the capsule to recapture the transcatheter heart valve prosthesis within the capsule, wherein during the distally advancing step, the distal end of the dampening arm is disposed between the leaflet and the interior surface of the frame to inhibit the leaflet from being pinched between struts of the frame during recapture of the transcatheter heart valve prosthesis.

Example 18. The method of any one of Examples 15 through 17, wherein the transcatheter heart valve prosthesis includes exactly three leaflets, the spindle includes exactly three dampening arms, and wherein the second end of each dampening arm is disposed between a corresponding one of the leaflets and the frame.

Example 19. The method of any one of Examples 15 through 18, wherein the dampening arm further comprises a pad coupled to the distal end of the dampening arm.

Example 20. The method of Example 19, wherein the pad includes a first portion and a second portion, wherein the first portion includes a covering material and the second portion is open to enable blood flow therethrough, wherein during the proximally retracting and further proximally retracting steps, the second portion of the pad enables blood flow therethrough.

Example 21. A method of loading a transcatheter heart valve prosthesis into a capsule of a delivery device, the method comprising: disposing a distal end of a dampening arm of a spindle between a leaflet of the transcatheter heart valve prosthesis and a frame of the transcatheter heart valve prosthesis; and radially compressing the transcatheter heart valve prosthesis within the capsule of the delivery device.

Example 22. The method of Example 21, further comprising coupling a proximal end of the dampening arm to a spindle body of the spindle.

Example 23. The method of Example 22, wherein coupling the proximal end of the dampening arm to the spindle body is after disposing the distal end of the dampening arm between the leaflet and the frame.

Example 24. The method of Example 21 or 22, wherein the spindle body comprises a cavity, wherein coupling the proximal end of the dampening arm of the spindle body comprises placing the proximal end of the dampening arm in the cavity and covering the cavity with the proximal end of the dampening arm disposed therein with a locking collar of the spindle.

Example 25. The method of Example 24, wherein the locking collar is disposed over a first portion of the spindle body with the cavity open and wherein covering the cavity comprises translating the locking collar to the cover the cavity.

Example 26. The method of claim any one of Example 21 through 25, wherein the spindle comprises exactly three dampening arms and the spindle body includes exactly three cavities equidistantly spaced apart from one another around a circumference of the spindle body.

Example 27. The method of any one of Examples 21 through 26, further comprising coupling the frame of the transcatheter heart valve prosthesis to the spindle.

Example 28. The method of Example 27, wherein coupling the frame of the transcatheter heart valve prosthesis to the spindle comprises placing a paddle extending from an end of the frame into a paddle pocket of the spindle body.

Example 29. The method of any one of Examples 19 through 28, wherein the dampening arm further includes a pad coupled to the distal end of the dampening arm.

Example 30. The method of Example 29, wherein a portion the pad is disposed outside of an outer surface of the frame.

1. A delivery assembly for percutaneously delivering a transcatheter heart valve prosthesis into a vasculature of a patient, the delivery assembly comprising:
a capsule configured to constrain the transcatheter heart valve prosthesis therein; and
a spindle including:
a spindle body coupled to a shaft disposed within the capsule; and
a dampening arm having a first end and a second end, the first end being coupled to the spindle body and the second end of the dampening arm being configured to extend between an interior surface of a frame and a leaflet of the transcatheter heart valve prosthesis to minimize leaflet protrusion through cells of the frame of the transcatheter heart valve prosthesis.

2. The delivery assembly of claim 1, further comprising a transcatheter heart valve prosthesis, the transcatheter heart valve prosthesis including a frame and a valve structure coupled to the frame, wherein the valve structure includes a plurality of leaflets, and wherein with the transcatheter heart valve prosthesis radially compressed within the capsule, the dampening arm extends within the frame with the second end of the dampening arm disposed radially outside of a leaflet of the plurality of leaflets.

3. The delivery assembly of claim 2, wherein the frame of the transcatheter heart valve prosthesis includes access cells disposed adjacent the plurality of leaflets of the valve structure, wherein the access cells are larger than other cells of the frame.

4. The delivery assembly of claim 2, wherein the transcatheter heart valve prosthesis includes exactly three leaflets and exactly three access cells, and the spindle includes exactly three dampening arms.

5. The delivery assembly of claim 1, wherein the spindle body further includes a cavity configured to receive the first end of the dampening arm.

6. The delivery assembly of claim 5, wherein the spindle body includes exactly three cavities equidistantly spaced apart from one another around a circumference of the spindle body.

7. The delivery assembly of claim 5, wherein the cavity is disposed at a distal portion of the spindle body such that a proximal portion of the spindle body does not contain the cavity.

8. The delivery assembly of claim 5, wherein the spindle further comprises a locking collar configured to secure the first end of the dampening arm to the spindle body.

9. The delivery assembly of claim 8, wherein spindle includes a first configuration wherein the locking collar is longitudinally spaced from the cavity such that the cavity is open to receive the first end of the damping arm and the spindle includes a second configuration wherein the locking collar surrounds the cavity with the first end of the dampening arm disposed in the cavity to couple the dampening arm to the spindle body.

10. The delivery assembly of claim 1, wherein the dampening arm further includes a pad coupled to the second end thereof.

11. The delivery assembly of claim 10, wherein the pad is substantially diamond-shaped.

12. The delivery assembly of claim 10, wherein the pad is substantially the same size as the access cell of the transcatheter heart valve prosthesis.

13. The delivery assembly of claim 12, wherein the pad comprises a mesh material.

14. The delivery assembly of claim 12, wherein a first portion of the pad includes a covering material and a second portion of the pad is open to enable blood flow therethrough.

15. A method of loading a transcatheter heart valve prosthesis into a capsule of a delivery device, the method comprising:
disposing a distal end of a dampening arm of a spindle between a leaflet of the transcatheter heart valve prosthesis and a frame of the transcatheter heart valve prosthesis; and
radially compressing the transcatheter heart valve prosthesis within the capsule of the delivery device.

16. The method of claim 15, further comprising coupling a proximal end of the dampening arm to a spindle body of the spindle.

17. The method of claim 16, wherein coupling the proximal end of the dampening arm to the spindle body is after disposing the distal end of the dampening arm between the leaflet and the frame.

18. The method of claim 15, wherein the spindle body comprises a cavity, wherein coupling the proximal end of the dampening arm of the spindle body comprises placing the proximal end of the dampening arm in the cavity and covering the cavity with the proximal end of the dampening arm disposed therein with a locking collar of the spindle.

19. The method of claim 18, wherein the locking collar is disposed over a first portion of the spindle body with the cavity open and wherein covering the cavity comprises translating the locking collar to the cover the cavity.

20. The method of claim 15, wherein the spindle comprises exactly three dampening arms and the spindle body includes exactly three cavities equidistantly spaced apart from one another around a circumference of the spindle body.

21-24. (canceled)
